(19) United States
(12) Patent Application Publication
(10) Pub. No.: US 2007/0298004 A1
(43) Pub. Date: Dec. 27, 2007

(54) Conditioning composition comprising asymmetric dialkyl quaternized ammonium salt

(75) Inventor: Yujun Li, Kobe (JP)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/820,550

(22) Filed: Jun. 20, 2007

Related U.S. Application Data
(60) Provisional application No. 60/815,420, filed on Jun. 21, 2006.

(51) Int. Cl. A61K 8/40; A61Q 5/12

(52) U.S. Cl. 424/70.19

(57) ABSTRACT

Disclosed is a conditioning composition comprising: an asymmetric di-alkyl ammonium salt cationic surfactant; a high melting point fatty compound; and an aqueous carrier. The composition of the present invention can provide improved ease-to-rinse feel while maintaining conditioning benefits.

This application claims the benefit of U. S. Provisional Application No. 60/815420, filed on Jun. 21, 2006.

### FIELD OF THE INVENTION

The present invention relates to a conditioning composition comprising: an asymmetric di-alkyl ammonium salt cationic surfactant; a high melting point fatty compound; and an aqueous carrier. The composition of the present invention can provide ease-to-rinse feel during and after rinsing while maintaining conditioning benefits such as slippery feel during the application to wet hair and softness and moisturized feel on dry hair.

### BACKGROUND OF THE INVENTION

A variety of approaches have been developed to condition the hair. A common method of providing conditioning benefit is through the use of conditioning agents such as cationic surfactants and polymers, high melting point fatty compounds, low melting point oils, silicone compounds, and mixtures thereof. Most of these conditioning agents are known to provide various conditioning benefits. For example, some cationic surfactants, when used together with some high melting point fatty compounds, are believed to provide a gel matrix which is suitable for providing a variety of conditioning benefits such as slippery feel during the application to wet hair and softness and moisturized feel on dry hair.

There is a need for hair conditioning compositions which provide improved ease-to-rinse feel, while maintaining conditioning benefits of gel matrix. Hair conditioning compositions containing a gel matrix provide slippery feel during rinsing the hair, even after rinsing the hair. Ease-to-rinse feel are, for example, fast reduced slippery feel, and/or increased clean feel after starting to rinse the hair. Such reduced slippery feel, and/or increased clean feel can be a signal for consumers to stop rinsing the hair conditioning compositions. Consumers who prefer such signals may keep rinsing the hair treated with hair conditioning composition by a large amount of water until they feel the signals. However, such rinsing activity provides less deposition of conditioning agents on the hair, thus less conditioning benefits to the dry hair.

One method of obtaining ease-to-rinse feel is the reduction of gel matrix and/or conditioning agents. However, such reduced gel matrix and/or conditioning agents provides reduced conditioning benefits, especially reduced gel matrix provides reduced slippery feel during the application.

Thus, there remains a need for hair conditioning compositions which provides such signals faster after starting to rinse the hair, while maintaining wet conditioning benefits of the gel matrix such as slippery feel during the application, and while remaining sufficient amount of deposition of conditioning agents on the hair.

Japanese Patent Laid-open H5-70327 discloses a hair cosmetic composition comprising an asymmetric dialkyl quaterized ammonium salt and one or more silicone components, the composition being said to provide excellent smoothness and softness, and have reduced oily residue perception and better application feel. Japanese Patent Laid-open H5-70327 also discloses, in Example 1, a hair cosmetic composition comprising: 1.5% of an asymmetric dialkyl quaterized ammonium salt; 0.5% of stearyl trimethyl ammonium chloride; 1.0% of a polyether modified silicone; and 3% of cetyl alcohol.

However, it has been found that; it is still not easy to obtain hair conditioning compositions which provide improved ease-to-rinse feel, while maintaining improved conditioning benefits such as slippery feel during the application to wet hair and softness and moisturized feel on dry hair.

Based on the foregoing, there remains a need for conditioning compositions which provide improved ease-to-rinse feel, while maintaining improved conditioning benefits such as slippery feel during the application to wet hair and softness and moisturized feel on dry hair.

None of the existing art provides all of the advantages and benefits of the present invention.

### SUMMARY OF THE INVENTION

The present invention is directed to a conditioning composition comprising by weight:
(a) from about 0.1% to about 10% of an asymmetric di-alkyl ammonium salt cationic surfactant having the formula:

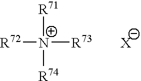

wherein R⁷¹ is selected from a non-functionalized straight alkyl group of from 12 to 30 carbon atoms, and R⁷² is selected from a non-functionalized branched alkyl group of from 6 to 10 carbon atoms; R⁷³ and R⁷⁴ are independently selected from an alkyl group of from 1 to about 4 carbon atoms or an aromatic, alkoxy, polyoxyalkylene, alkylamido, hydroxyalkyl, aryl or alkylaryl group having up to about 4 carbon atoms; and X- is a salt-forming anion such as those selected from halogen, acetate, citrate, lactate, glycolate, phosphate, nitrate, sulfonate, sulfate, alkylsulfate, and alkyl sulfonate radicals;
(b) from about 1% to about 15% of a high melting point fatty compound; and
(c) an aqueous carrier.

The present invention is also directed to a conditioning composition comprising by weight:
(a) from about 0. 1% to about 10% of an asymmetric di-alkyl ammonium salt cationic surfactant having the formula:

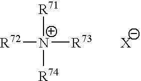

wherein R⁷¹ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; R⁷² is selected from an alkyl group of from 5 to 12 carbon atoms or an aromatic, alkoxy, polyoxyalkylene, alkylamido, hydroxyalkyl, aryl or alkylaryl group having up to about 12 carbon atoms; R⁷³ and R⁷⁴ are independently selected from an alkyl group of from 1 to about 4 carbon atoms or an aromatic, alkoxy, polyoxyalkylene, alkylamido, hydroxyalkyl, aryl or alkylaryl group having up to about 4 carbon atoms; and X- is a salt-forming anion such as those selected from halogen, acetate, citrate, lactate, glycolate, phosphate, nitrate, sulfonate, sulfate, alkylsulfate, and alkyl sulfonate radicals;
(b) from about 1% to about 15% of a high melting point fatty compound;
(c) an aqueous carrier; and
(d) from about 0.1% to about 9% of an additional cationic surfactant selected from mono-alkyl cationic surfactants; and
   wherein the weight ratio of the asymmetric dialkyl quaternized ammonium salt cationic surfactant to the additional cationic surfactant is within the range of from about 1:1 to about 1:10.

The present invention is further directed to a conditioning composition comprising by weight:
(a) from about 0.1% to about 10% of an asymmetric di-alkyl ammonium salt cationic surfactant having the formula:

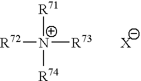

wherein R⁷¹ is selected from a non-functionalized straight alkyl group of from 12 to 30 carbon atoms, and R⁷² is selected from a non-functionalized branched alkyl group of from 6 to 10 carbon atoms; R⁷³ and R⁷⁴ are independently selected from an alkyl group of from 1 to about 4 carbon atoms or an aromatic, alkoxy, polyoxyalkylene, alkylamido, hydroxyalkyl, aryl or alkylaryl group having up to about 4 carbon atoms; and X⁻ is a salt-forming anion such as those selected from halogen, acetate, citrate, lactate, glycolate, phosphate, nitrate, sulfonate, sulfate, alkylsulfate, and alkyl sulfonate radicals;
(b) from about 1% to about 15% of a high melting point fatty compound;
(c) an aqueous carrier; and
(d) from about 0.1% to about 9% of an additional cationic surfactant selected from mono-alkyl cationic surfactants; and
   wherein the weight ratio of the asymmetric dialkyl quaternized ammonium salt cationic surfactant to the additional cationic surfactant is within the range of from about 1:1 to about 1:10.

The conditioning compositions of the present invention can provide improved ease-to-rinse feel, while maintaining conditioning benefits such as slippery feel during the application to wet hair and softness and moisturized feel on dry hair.

These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

### Composition

It is believed that; the asymmetric dialkyl quaternized ammonium salt cationic surfactants can form loose gel matrix which is sufficient to provide wet conditioning benefits and which can provide ease-to-rinse feel. It is also believed that, this loose gel matrix is different from gel matrix obtained with other cationic surfactants. For example, mono-alkyl cationic surfactants having one long alkyl chain are believed to form well-packed gel matrix which provides wet conditioning benefits but also provides reduced ease-to-rinse feel, and di-alkyl cationic surfactants having two long alkyl chains are believed to form reduced gel matrix which provide reduced wet conditioning benefits.

Preferably, when containing cationic surfactants and/or gel matrix formed by cationic surfactants and high melting point fatty compounds, the composition of the present invention is substantially free of anionic surfactants and anionic polymers, in view of avoiding undesirable interaction with cationic surfactants and/or in view of stability of the gel matrix. In the present invention, “substantially free of anionic surfactants and anionic polymers” means that the composition contains 1% or less, preferably 0.5% or less, more preferably totally 0% of total of anionic surfactants and anionic polymers.

### Asymetric Dialkyl Quaternized Ammonium Salt Cationic Surfacants

The composition of the present invention comprises an asymmetric dialkyl quaternized ammonium salt cationic surfactant.

The asymmetric dialkyl quaternized ammonium salt cationic surfactant is included in the composition at a level by weight of from about 0.1% to about 10%, preferably from about 0.2% to about 5%, more preferably from about 0.4% to about 3% in view of balance between ease-to-rinse feel and wet conditioning benefits. The use of higher level of asymmetric dialkyl quatemized ammonium salt tends to lead to reduced wet conditioning benefits such as reduced slippery feel, while the use of lower level of asymmetric dialkyl quaternized ammonium salt tends to lead to reduced ease-to-rinse feel.

The asymmetric dialkyl quaternized ammonium salt cationic surfactants useful herein are those having the formula (I):

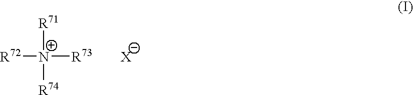

wherein R⁷¹ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; R⁷² is selected from an alkyl group of from 5 to 12 carbon atoms or an aromatic, alkoxy, polyoxyalkylene, alkylamido, hydroxyalkyl, aryl or alkylaryl group having up to about 12 carbon atoms; R⁷³ and R⁷⁴ are independently selected from an alkyl group of from 1 to about 4 carbon atoms or an aromatic, alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether linkages, and other groups such as amino groups. The longer chain alkyl groups, e.g., those of about 12 carbons, or higher, can be saturated or unsaturated and/or straight or branched. Preferably, R⁷¹ is selected from a non-functionalized alkyl group of from 12 to 30 carbon atoms, preferably from 16 to 22 carbon atoms, more preferably 18 to 22 carbon atoms, still more preferably 18 carbon atoms; R⁷² is selected from a non-functionalized alkyl group of from 5 to 12 carbon atoms, more preferably from 6 to 10 carbon atoms, still more preferably 8 carbon atoms; R⁷³ and R⁷⁴ are independently selected from CH₃, C₂H₅, C₂H₄OH, and mixtures thereof; and X is selected from the group consisting of Cl, Br, CH₃OSO₃, C₂H₅OSO₃, and mixtures thereof. More preferably, R⁷¹ is a straight, saturated non-functionalized alkyl group, and R⁷² is a branched saturated non-functionalized alkyl group. Still more preferably, the branched group of R⁷² is a straight, saturated alky group of from 1 to 4 carbon atoms, even more preferably 2 carbon atoms.

It has been surprisingly found that; the above asymmetric dialkyl quaternized ammonium salt cationic surfactants provide improved ease-to-rinse feel compared to mono-alkyl quaternized ammonium salt cationic surfactants such as behenyl trimethyl ammonium salts and symmetric dialkyl quaternized ammonium cationic surfactants such as distearyl dimethyl ammonium salts, while still maintaining balanced wet conditioning benefits like slippery feel. It has been further found that; among the above asymmetric dialkyl quaternized ammonium salt cationic surfactants, those having a longer straight saturated alkyl group and a shorter branched alkyl group, together with two more C1-4 alkyl groups, provide improved balance between ease-to-rinse feel and wet conditioning benefits, compared to other asymmetric dialkyl quaternized ammonium salt cationic surfactants such as those having a longer branched alkyl group and a shorter straight alkyl group together with two more C1-4 alkyl groups. Furthermore, it has been found that; among the above asymmetric dialkyl quaternized ammonium salt cationic surfactants having a longer straight saturated alkyl group and a shorter branched alkyl group, those having the shorter branched alkyl of from 6 to 10 carbon atoms, preferably 8 carbon atoms provide further improved balance between ease-to-rinse feel and wet conditioning benefits, compared to those having the shorter branched alkyl of more than 11 carbon atoms.

It is believed that the use of alkylsulfate such as methosulfate and ethosulfate as a salt-forming anion may be able to provide better conditioning benefits especially wet conditioning benefits, compared to other salt-forming anions.

Nonlimiting examples of preferred asymmetric dialkyl quaternized ammonium salt cationic surfactants include: stearyl ethylhexyl dimonium methosulfate available, for example, with tradename Arquad HTL8-MS from Akzo Nobel having the following structure:

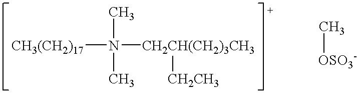

### Additional Cationic Surfactants

The composition of the present invention preferably contains an additional cationic surfactant other than the asymmetric dialkyl quaternized ammonium salt cationic surfactants. The additional cationic surfactant is included in the composition at a level by weight of from about 0.1% to about 9%, preferably from about 0.2% to about 5%, more preferably from about 0.4% to about 3%, in view of balance between ease-to-rinse feel and wet conditioning benefits. When the additional cationic surfactant is contained in the composition of the present invention, a total amount of cationic surfactants is preferably from about 0.2% to about 10%, more preferably from about 0.5% to about 8%, still more preferably from about 1% to about 5%, even more preferably from about 1.5% to about 4%. When the additional cationic surfactant is contained in the composition of the present invention, it is preferred to use the additional cationic surfactant at a level such that the weight ratio of the asymmetric dialkyl quatemized ammonium salt cationic surfactant to the additional cationic surfactant is within the range of from about 1:1 to about 1:10, more preferably from about 1:1 to about 1:5, still more preferably from about 1:1 to about 1:3, in view of balance between ease-to-rinse feel and wet conditioning benefits. It is also believed that, when the additional cationic surfactant is contained in the composition at the above weight ratio to the asymmetric dialkyl quatemized ammonium salt cationic surfactant, the composition can provide improved deposition of ingredients included therein, especially silicones when included.

The additional cationic surfactants useful herein are mono-alkyl cationic surfactants having one long alkyl chain which has from 12 to 22 carbon atoms, preferably from 16 to 22 carbon atoms, more preferably C18-22 alkyl group, in view of providing balanced wet conditioning benefits. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms. Such mono-alkyl cationic surfactants include, for example, mono-alkyl quaternary ammonium salts and mono-alkyl amines. Mono-alkyl quaternary ammonium salts include, for example, those having a non-functionalized long alkyl chain and those having a functionalized long alkyl chain such as those having an ester-linkage. Mono-alkyl amines include, for example, mono-alkyl amidoamines and salts thereof.

In the present invention, the above mono-alkyl cationic surfactants are preferred compared to other cationic surfactants such as di- and tri-alkyl cationic surfactants having two or three long-alkyl chains, in view of providing desired gel matrix and wet conditioning benefits.

It is believed that; mono-alkyl cationic surfactants having a longer alkyl group provide improved deposition on the hair, thus can provide improved conditioning benefits such as improved softness on dry hair, compared to cationic surfactant having a shorter alkyl group. It is also believed that such cationic surfactants can provide reduced irritation, compared to cationic surfactants having a shorter alkyl group.

It is also believed that the use of alkylsulfate such as methosulfate and ethosulfate as a salt-forming anion may be able to provide better conditioning benefits especially wet conditioning benefits, compared to other salt-forming anions.

Mono-long alkyl quaternized ammonium salts useful herein are those having the formula (II):

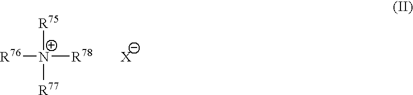

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 22 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

Mono-alkyl amines are also suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylaamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid. The amines herein are preferably partially neutralized with any of the acids at a molar ratio of the amine to the acid of from about 1:0.3 to about 1:2, more preferably from about 1:0.4 to about 1:1.

### High Melting Point Fatty Compound

The composition of the present invention comprises a high melting point fatty compound. The high melting point fatty compound is included in the composition at a level of from about 1% to about 15%, preferably from about 3% to about 10%, more preferably from about 5% to about 8% by weight of the composition, in view of providing improved conditioning benefits such as slippery feel during the application to wet hair, softness and moisturized feel on dry hair.

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about 22 carbon atoms. These fatty alcohols are saturated and can be straight or branched chain alcohols. Preferred fatty alcohols include, for example, cetyl alcohol, stearyl alcohol, behenyl alcohol, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

### Aqueous Carrier

The conditioning composition of the present invention comprises an aqueous carrier. The level and species of the carrier are selected according to the compatibility with other components, and other desired characteristic of the product. Generally, the compositions of the present invention comprise from about 20% to about 99%, preferably from about 30% to about 95%, and more preferably from about 80% to about 95% water.

The carrier useful in the present invention includes water and water solutions of lower alkyl alcohols and polyhydric alcohols. The lower alkyl alcohols useful herein are monohydric alcohols having 1 to 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product.

### Gel Matrix

Preferably, the above cationic surfactants, together with high melting point fatty compounds and an aqueous carrier, form a gel matrix in the composition of the present invention.

The gel matrix is suitable for providing various conditioning benefits such as slippery feel during the application to wet hair and softness and moisturized feel on dry hair. In view of providing the above gel matrix, the cationic surfactant and the high melting point fatty compound are contained at a level such that the weight ratio of the cationic surfactant to the high melting point fatty compound is in the range of, preferably from about 1:1 to about 1:10, more preferably from about 1:1 to about 1:6.

For forming gel matrix, it is preferred to prepare the composition by the following method:

Water is typically heated to at least about 70° C., preferably between about 80° C. and about 90° C. The cationic surfactant and the high melting point fatty compound are combined with the water to form a mixture. The temperature of the mixture is preferably maintained at a temperature higher than both the melting temperature of the cationic surfactant and the melting temperature of the high melting point fatty compound, and the entire mixture is homogenized. After mixing until no solids are observed, the mixture is gradually cooled (e.g., at a rate of from about 1° C./minute to about 5° C./minute) to a temperature below 60° C., preferably less than about 55° C. During this gradual cooling process, a significant viscosity increase is observed at between about 55° C. and about 65° C. This indicates the formation of gel matrix. The high molecular weight water-soluble cationic polymer can be added to the mixture with agitation at about 55° C., or prior to the cooling down. Additional components are then combined with the gel matrix, and cooled to room temperature.

Preferably, the present invention comprises, by weight of the composition, from about 60% to about 99%, preferably from about 70% to about 95%, and more preferably from about 80% to about 95% of a gel matrix, to which optional ingredients such as silicones can be added. The composition containing the above amount of gel matrix is typically characterized by a viscosity of from about 5,000 cps to about 40,000 cps, preferably from about 10,000 cps to about 30,000 cps, and more preferably from about 12,000 cps to about 28,000 cps, as measured at 25° C., by means of a Brookfield Viscometer at shear rate of 1.0 rpm. Although the composition of the present invention may contain a thickening polymer, the composition of the present invention can have the above viscosity without the presence of any thickening polymer.

### Silicone Compound

Preferably, the compositions of the present invention contain a silicone compound. It is believed that the silicone compound can provide smoothness and softness on dry hair. The silicone compounds herein can be used at levels by weight of the composition of preferably from about 0.1% to about 20%, more preferably from about 0.15% to about 10%, still more preferably from about 0.2% to about 8%.

The silicone compounds useful herein, as a single compound, as a blend or mixture of at least two silicone compounds, or as a blend or mixture of at least one silicone compound and at least one solvent, have a viscosity of preferably from about 1,000 to about 2,000,000mPa·s at 25° C.

The viscosity can be measured by means of a glass capillary viscometer as set forth in Dow Coming Corporate Test Method CTM0004, Jul. 20, 1970. Suitable silicone fluids include polyalkyl siloxanes, polyaryl siloxanes, polyalkylaryl siloxanes, polyether siloxane copolymers, amino substituted silicones, quaternized silicones, and mixtures thereof. Other nonvolatile silicone compounds having conditioning properties can also be used.

Preferably, the silicone compounds have an average particle size of from about 1 microns to about 50 microns, in the composition.

The silicone compounds useful herein include polyalkyl or polyaryl siloxanes with the following structure:

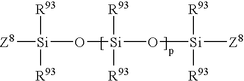

wherein R⁹³ is alkyl or aryl, and p is an integer from about 7 to about 8,000. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the hair, is compatible with the other components of the composition, is chemically stable under normal use and storage conditions, and is capable of being deposited on and conditions the hair. Suitable Z⁸ groups include hydroxy, methyl, methoxy, ethoxy, propoxy, and aryloxy. The two R⁹³ groups on the silicon atom may represent the same group or different groups. Preferably, the two R⁹³ groups represent the same group. Suitable R⁹³ groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their Viscasil® and TSF 451 series, and from Dow Corning in their Dow Corning SH200 series.

The above polyalkylsiloxanes are available, for example, as a mixture with silicone compounds having a lower viscosity. Such mixtures have a viscosity of preferably from about 1,000 mPa·s to about 100,000 mPa·s, more preferably from about 5,000 mPa·s to about 50,000 mPa·s. Such mixtures preferably comprise: (i) a first silicone having a viscosity of from about 100,000 mPa·s to about 30,000,000 mPa·s at 25° C., preferably from about 100,000 mPa·s to about 20,000,000 mPa·s; and (ii) a second silicone having a viscosity of from about 5 mPa·s to about 10,000 mPa·s at 25° C., preferably from about 5 mPa·s to about 5,000 mPa·s. Such mixtures useful herein include, for example, a blend of dimethicone having a viscosity of 18,000,000 mPa·s and dimethicone having a viscosity of 200 mPa·s available from GE Toshiba, and a blend of dimethicone having a viscosity of 18,000,000 mPa·s and cyclopentasiloxane available from GE Toshiba.

The silicone compounds useful herein also include a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. The silicone gums are available, for example, as a mixture with silicone compounds having a lower viscosity. Such mixtures useful herein include, for example, Gum/Cyclomethicone blend available from Shin-Etsu.

The silicone compounds that can be used include, for example, a polypropylene oxide modified polydimethylsiloxane although ethylene oxide or mixtures of ethylene oxide and propylene oxide can also be used. The ethylene oxide and polypropylene oxide level should be sufficiently low so as not to interfere with the dispersibility characteristics of the silicone. These materials are also known as dimethicone copolyols.

Silicone compounds useful herein also include amino substituted materials. Preferred aminosilicones include, for example, those which conform to the general formula (III):

(R₁)ₐG₃₋ₐ-Si—(—OSiG₂)ₙ—(—OSiG_{b}(R₁)_{2-b})ₘ—O—SiG₃₋ₐ(R₁)ₐ

wherein G is hydrogen, phenyl, hydroxy, or C₁-C₈ alkyl, preferably methyl; a is 0 or an integer having a value from 1 to 3, preferably 1; b is 0, 1 or 2, preferably 1; n is a number from 0 to 1,999, preferably from about to about 100 to 2,000, more preferably from 300 to 1,800; m is an integer from 0 to 1,999, preferably m is 0; the sum of n and m is a number from 1 to 2,000; a and m are not both 0; R₁ is a monovalent radical conforming to the general formula CqH_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical, preferably an alkyl radical from about C₁ to about C₂₀; A⁻ is a halide ion. L is preferably —N(CH₃)₂ or —NH₂.

One highly preferred amino silicones are those corresponding to formula (III) wherein m=0, a=1, q=3, G=methyl, n is preferably from about 1500 to about 1700, more preferably 1600; and L is —N(CH₃)₂ or —NH₂, more preferably —NH₂. Another highly preferred amino silicones are those corresponding to formula (III) wherein m=0, a=1, q=3, G=methyl, n is preferably from about 400 to about 600, more preferably about 500; and L is —N(CH₃)₂ or —NH₂, more preferably —NH₂. Such highly preferred amino silicones can be called as terminal aminosilicones, as one or both ends of the silicone chain are terminated by nitrogen containing group.

The above aminosilicones, when incorporated into the composition, can be mixed with solvent having a lower viscosity. Such solvents include, for example, polar or non-polar, volatile or non-volatile oils. Such oils include, for example, silicone oils, hydrocarbons, and esters. Among such a variety of solvents, preferred are those selected from the group consisting of non-polar, volatile hydrocarbons, volatile cyclic silicones, non-volatile linear silicones, and mixtures thereof. The non-volatile linear silicones useful herein are those having a viscosity of from about 1 to about 20,000 centistokes, preferably from about 20 to about 10,000 centistokes at 25° C. Among the preferred solvents, highly preferred are non-polar, volatile hydrocarbons, especially non-polar, volatile isoparaffins, in view of reducing the viscosity of the aminosilicones and providing improved hair conditioning benefits such as reduced friction on dry hair. Such mixtures have a viscosity of preferably from about 1,000 mPa·s to about 100,000 mPa·s, more preferably from about 5,000 mPa·s to about 50,000 mPa·s.

Other suitable alkylamino substituted silicone compounds include those represented by the following structure:

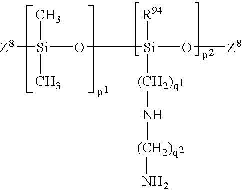

wherein R⁹⁴ is H, CH₃ or OH; p¹ and p² are integers of 1 or above, and wherein sum of p¹ and p² is from 650 to 1,500; q¹ and q² are integers of from 1 to 10. Z⁸ represents groups which block the ends of the silicone chains. Suitable Z⁸ groups include hydroxy, methyl, methoxy, ethoxy, propoxy, and aryloxy. Highly preferred are those known as “amodimethicone”. Commercially available amodimethicones useful herein include, for example, BY16-872 available from Dow Corning.

Other amino substituted silicone polymers which can be used are represented by the formula:

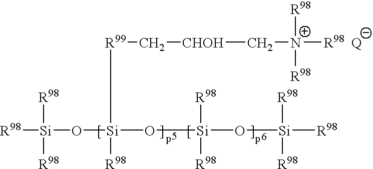

wherein R⁹⁸ denotes a monovalent hydrocarbon radical having from 1 to 18 carbon atoms, preferably an alkyl or alkenyl radical such as methyl; R⁹⁹ denotes a hydrocarbon radical, preferably a C₁-C₁₈ alkylene radical or a C₁-C₁₈, and more preferably C₁-C₈, alkyleneoxy radical; Q⁻ is a halide ion, preferably chloride; p5 denotes an average statistical value from 2 to 20, preferably from 2 to 8; p⁶ denotes an average statistical value from 20 to 200, and preferably from 20 to 50.

The silicone compounds may further be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and mixtures thereof.

### Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: low melting point oils having a melting point of less than 25° C. including, for example, unsaturated fatty alcohols such as oleyl alcohol and ester oils such as pentaerythritol ester oils; cationic conditioning polymers including, for example, cationic celluloses and cationic guar gums; polyethylene glycols; other conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; perfumes; and sequestering agents, such as disodium ethylenediamine tetra-acetate; ultraviolet and infrared screening and absorbing agents such as octyl salicylate, octyl methoxycinnamate, benzophenone-3 and nenzophenone-4; and antidandruff agents such as zinc pyrithione and salicylic acid.

### Product Forms

The conditioning compositions of the present invention can be in the form of rinse-off products or leave-on products, and can be formulated in a wide variety of product forms, including but not limited to creams, gels, emulsions, mousses and sprays.

The conditioning composition of the present invention is especially suitable for rinse-off hair conditioner. Such compositions are preferably used by following steps:
(i) after shampooing hair, applying to the hair an effective amount of the conditioning compositions for conditioning the hair; and
(ii) then rinsing the hair.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

[Compositions]

| Components | Ex. 1 | Ex. 2 | Ex. 3 | Ex. 4 | Ex. 5 | Ex. 6 | Ex. 7 | EX. 8 |
|---|---|---|---|---|---|---|---|---|
| Asymmetric dialkyl quaternized | 0.9 | 0.15 | 0.9 | 1.8 | 0.9 | 0.9 | 0.9 | 0.9 |
| ammonium salt cationic surfactant-1 *1 | | | | | | | | |
| Behenyl trimethyl ammonium chloride | 1.4 | 0.23 | — | — | — | — | 1.4 | 1.4 |
| Behenyl trimethyl ammonium methosulfate | — | — | 1.4 | — | — | — | — | — |
| Stearyl trimethyl ammonium chloride | — | — | — | 2.8 | — | — | — | — |
| Behenamidopropyl Dimethylamine | — | — | — | — | 1.4 | — | — | — |
| Stearamidopropyl Dimethylamine | — | — | — | — | — | 1.4 | — | — |
| L-Glutamic acid | — | — | — | — | 0.38 | 0.44 | — | — |
| Cetyl alcohol | 1.9 | 0.32 | 1.9 | 3.8 | 1.9 | 1.9 | 1.9 | 1.9 |
| Stearyl alcohol | 4.6 | 0.77 | 4.6 | 9.2 | 4.6 | 4.6 | 4.6 | 3.6 |
| Behenyl alcohol | — | — | — | — | — | — | — | 1.0 |
| Dimethicone blend *2 | — | — | — | — | — | — | 2.0 | — |
| Dimethicone/Cyclomethicone *3 | — | — | — | — | — | — | — | 4.2 |
| Aminosilicone *4 | 1.5 | 0.5 | 1.0 | 1.5 | 0.5 | 0.5 | — | — |
| Isopropanol | 0.3 | 0.15 | 0.3 | 0.6 | 0.3 | 0.3 | 0.3 | 0.3 |
| Disodium EDTA | 0.13 | 0.13 | 0.13 | 0.13 | 0.13 | 0.13 | 0.13 | 0.13 |
| Benzyl alcohol | 0.4 | 0.4 | 0.4 | 0.4 | 0.4 | 0.4 | 0.4 | 0.4 |
| Kathon CG *5 | 0.033 | 0.033 | 0.033 | 0.033 | 0.033 | 0.033 | 0.033 | 0.033 |
| Perfume | 0.40 | 0.40 | 0.40 | 0.40 | 0.40 | 0.40 | 0.40 | 0.40 |
| Panthenol | 0.03 | 0.03 | 0.03 | — | 0.03 | 0.03 | 0.03 | 0.03 |
| Panthenyl ethyl ether | 0.03 | 0.03 | 0.03 | — | 0.03 | 0.03 | 0.03 | 0.03 |
| Sodium Hydroxide | 0.014 | 0.014 | 0.014 | 0.014 | 0.014 | 0.014 | 0.014 | 0.014 |

| Deionized Water | q.s. to 100% | | | | | | |
|---|---|---|---|---|---|---|---|
| Components | Ex. 9 | Ex. 10 | Ex. 11 | Ex. 12 | Ex. 13 | Ex. 14 | Ex. 15 |
| Asymmetric dialkyl quaternized ammonium | 0.5 | 0.6 | 0.7 | 1.0 | 2.0 | 0.9 | 1.8 |
| salt cationic surfactant-1 *1 | | | | | | | |
| Behenyl trimethyl ammonium chloride | 0.8 | 0.9 | — | — | — | — | — |
| Behenyl trimethyl ammonium methosulfate | — | — | 1.1 | 1.5 | 3.0 | 1.4 | — |
| Cetyl alcohol | 1.1 | 1.3 | 1.5 | 1.9 | 3.8 | 1.9 | 1.9 |
| Stearyl alcohol | 2.8 | 3.2 | 3.7 | 4.6 | 9.2 | 4.6 | 4.6 |
| Aminosilicone *4 | 0.5 | 0.5 | 0.5 | 1.5 | 1.0 | 1.5 | 0.5 |
| Isopropanol | 0.2 | 0.2 | 0.2 | 0.3 | 0.6 | 0.3 | — |
| Disodium EDTA | 0.13 | 0.13 | 0.13 | 0.13 | 0.13 | 0.13 | 0.13 |
| Benzyl alcohol | 0.4 | 0.4 | 0.4 | 0.4 | 0.4 | 0.4 | 0.4 |
| Kathon CG *5 | 0.033 | 0.033 | 0.033 | 0.033 | 0.033 | 0.033 | 0.033 |
| Perfume | 0.40 | 0.40 | 0.40 | 0.40 | 0.40 | 0.40 | 0.40 |
| Panthenol | — | 0.03 | — | — | 0.03 | 0.03 | 0.03 |
| Panthenyl ethyl ether | — | 0.03 | — | — | 0.03 | 0.03 | 0.03 |
| Vitamin E | 0.01 | — | 0.01 | 0.01 | — | — | — |
| Octyl methoxycinnamate | — | — | — | 0.09 | — | — | — |
| Benzophenone-3 | — | — | — | 0.09 | — | — | — |

| | |
|---|---|
| Deionized Water | q.s. to 100% |

Definitions of Components
*1 Asymmetric dialkyl quaternized ammonium salt cationic surfactant-1: Stearyl ethylhexyl dimonium methosulfate, available with a tradename Arquad HTL8-MS from Akzo Nobel
*2 Dimethicone blend: a blend of dimethicone having a viscosity of 18,000,000 mPa · s and dimethicone having a viscosity of 200 mPa · s available from GE Toshiba
*3 Dimethicone/Cyclomethicone: a blend dimethicone having a viscosity of 18,000,000 mPa · s and cyclopentasiloxane available from GE Toshiba
*4 Aminosilicone: Terminal aminosilicone which is available from GE having a viscosity 10,000 mPa · s, and having following formula (III): (R₁)ₐG₃₋ₐ-Si—(—OSiG₂₎ₙ-(-OSiG_{b}(R₁)_{2-b})ₘ—O—SiG₃₋ₐ(R₁)ₐ (III)wherein G is methyl; a is an integer of 1; n is a number from 400 to about 600; m is an integer of 0; R₁ is a monovalent radical conforming to the general formula CqH_{2q}L, wherein q is an integer of 3 and L is-NH₂
*5 Kathon CG: Available from Rohm & Haas

### Method of Preparation

The conditioning compositions of “Ex. 1” through “Ex. 15” as shown above can be prepared by any conventional method well known in the art. They are suitably made as follows:

Cationic surfactants and high melting point fatty compounds are added to water with agitation, and heated to about 80° C. The mixture is cooled down to about 55° C. If included, silicone compounds, perfumes, preservatives are added to the mixture with agitation. Then the mixture is cooled down to room temperature.

Examples 1 through 15 are hair conditioning compositions of the present invention which are particularly useful for rinse-off use. The embodiments disclosed and represented by the previous “Ex. 1” through “Ex. 15” have many advantages. For example, they can provide improved ease-to-rinse feel, while maintaining improved conditioning benefits of gel matrix such as slippery feel during the application to wet hair. They can also provide such ease-to-rinse feel while remaining a sufficient amount of deposition of conditioning agents on the hair to provide dry conditioning benefits such as softness and moisturized feel on dry hair.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm,” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the same term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A conditioning composition comprising by weight:
(a) from about 0.1% to about 10% of an asymmetric di-alkyl ammonium salt cationic surfactant having the formula: 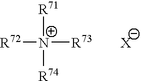
wherein R⁷¹ is selected from a non-functionalized straight alkyl group of from 12 to 30 carbon atoms, and R⁷² is selected from a non-functionalized branched alkyl group of from 6 to 10 carbon atoms; R⁷³ and R⁷⁴ are independently selected from an alkyl group of from 1 to about 4 carbon atoms or an aromatic, alkoxy, polyoxyalkylene, alkylamido, hydroxyalkyl, aryl or alkylaryl group having up to about 4 carbon atoms; and X- is a salt-forming anion such as those selected from halogen, acetate, citrate, lactate, glycolate, phosphate, nitrate, sulfonate, sulfate, alkylsulfate, and alkyl sulfonate radicals;
(b) from about 1% to about 15% of a high melting point fatty compound; and
(c) an aqueous carrier.

2. A conditioning composition comprising by weight:
(a) from about 0.1% to about 10% of an asymmetric di-alkyl ammonium salt cationic surfactant having the formula: 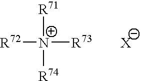
wherein R⁷¹ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; R⁷² is selected from an alkyl group of from 5 to 12 carbon atoms or an aromatic, alkoxy, polyoxyalkylene, alkylamido, hydroxyalkyl, aryl or alkylaryl group having up to about 12 carbon atoms; R⁷³ and R⁷⁴ are independently selected from an alkyl group of from 1 to about 4 carbon atoms or an aromatic, alkoxy, polyoxyalkylene, alkylamido, hydroxyalkyl, aryl or alkylaryl group having up to about 4 carbon atoms; and X- is a salt-forming anion such as those selected from halogen, acetate, citrate, lactate, glycolate, phosphate, nitrate, sulfonate, sulfate, alkylsulfate, and alkyl sulfonate radicals;
(b) from about 1% to about 15% of a high melting point fatty compound;
(c) an aqueous carrier; and
(d) from about 0.1% to about 9% of an additional cationic surfactant selected from mono-alkyl cationic surfactants; and
wherein the weight ratio of the asymmetric dialkyl quaternized ammonium salt cationic surfactant to the additional cationic surfactant is within the range of from about 1:1 to about 1:10.

3. A conditioning composition comprising by weight:
(a) from about 0.1% to about 10% of an asymmetric di-alkyl ammonium salt cationic surfactant having the formula: 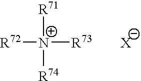
wherein R⁷¹ is selected from a non-functionalized straight alkyl group of from 12 to 30 carbon atoms, and R72 is selected from a non-functionalized branched alkyl group of from 6 to 10 carbon atoms; R⁷³ and R⁷⁴ are independently selected from an alkyl group of from 1 to about 4 carbon atoms or an aromatic, alkoxy, polyoxyalkylene, alkylamido, hydroxyalkyl, aryl or alkylaryl group having up to about 4 carbon atoms; and X⁻ is a salt-forming anion such as those selected from halogen, acetate, citrate, lactate, glycolate, phosphate, nitrate, sulfonate, sulfate, alkylsulfate, and alkyl sulfonate radicals;
(b) from about 1% to about 15% of a high melting point fatty compound;
(c) an aqueous carrier; and
(d) from about 0.1% to about 9% of an additional cationic surfactant selected from mono-alkyl cationic surfactants; and
wherein the weight ratio of the asymmetric dialkyl quaternized ammonium salt cationic surfactant to the additional cationic surfactant is within the range of from about 1:1 to about 1:10.

4. The conditioning composition of claim 2 wherein the weight ratio of the asymmetric dialkyl quaternized ammonium salt cationic surfactant to the additional cationic surfactant is within the range of from about 1:1 to about 1:5,

5. The conditioning composition of claim 2 wherein the weight ratio of the asymmetric dialkyl quaternized ammonium salt cationic surfactant to the additional cationic surfactant is within the range of from about 1:1 to about 1:3

6. The conditioning composition of claims 1 wherein X⁻ is an alkylsulfate selected from methosulfate or ethosulfate.

7. The conditioning composition of claims 1 further comprising from about 0.1% to about 20% of a silicone compound.

8. The conditioning composition of claims 1 wherein the composition is substantially free of anionic surfactants and anionic polymers.

9. The conditioning composition of claims 1 which is a rinse-off hair conditioning composition.

10. The conditioning composition of claim 3 wherein the weight ratio of the asymmetric dialkyl quaternized ammonium salt cationic surfactant to the additional cationic surfactant is within the range of from about 1:1 to about 1:5.

11. The conditioning composition of claim 3 wherein the weight ratio of the asymmetric dialkyl quaternized ammonium salt cationic surfactant to the additional cationic surfactant is within the range of from about 1:1 to about 1:3.

12. The conditioning composition of claim 2 wherein X⁻ is an alkylsulfate selected from methosulfate or ethosulfate.

13. The conditioning composition of claim 3 wherein X⁻ is an alkylsulfate selected from methosulfate or ethosulfate.

14. The conditioning composition of claim 2 further comprising from about 0.1% to about 20% of a silicone compound.

15. The conditioning composition of claim 3 further comprising from about 0.1% to about 20% of a silicone compound.

16. The conditioning composition of claim 2 wherein the composition is substantially free of anionic surfactants and anionic polymers.

17. The conditioning composition of claim 3 wherein the composition is substantially free of anionic surfactants and anionic polymers.

18. The conditioning composition of claim 2 which is a rinse-off hair conditioning composition.

19. The conditioning composition of claim 3 which is a rinse-off hair conditioning composition.
